(19) United States
(12) Patent Application Publication
(10) Pub. No.: US 2025/0073489 A1
(43) Pub. Date: Mar. 6, 2025

(54) PHOTOTHERAPY DEVICE AND PHOTOTHERAPY APPARATUS FOR CEREBRAL FUNCTIONAL RELATED DISEASE

(71) Applicant: DANYANG HUICHUANG MEDICAL EQUIPMENT CO., LTD., Jiangsu (CN)

(72) Inventor: Daifa WANG, Jiangsu (CN)

(21) Appl. No.: 18/724,037

(22) PCT Filed: Dec. 28, 2022
(86) PCT No.: PCT/CN2022/142916

§ 371 Date: Jun. 25, 2024

(30) Foreign Application Priority Data
Dec. 28, 2021 (CN) 202111624718.4
Jul. 26, 2022 (CN) 202210886242.X

(51) Int. Cl. A61N 5/06; A61N 5/00

(52) U.S. Cl. CPC A61N 5/0618; A61N 2005/007; A61N 2005/0652; A61N 2005/0659

(57) ABSTRACT

The embodiments of the disclosure provide a phototherapy device and a phototherapy apparatus for cerebral functional related diseases. The phototherapy device comprises a headgear, irradiation assembly and a cooled gas supply mechanism. The headgear includes a housing assembly, inner side of which forms a spaced cavity with scalp of a user and is provided with a first gas inlet, the spaced cavity is constructed to form gas pathway between the first gas inlet and the scalp of the user. The irradiation assembly is mounted to the housing assembly with its light emergent side located to the inner side of the housing assembly. The cooled gas supply mechanism is interconnected to the first gas inlet through the housing assembly and supplies the cooled gas for passing through the first gas inlet and the spaced cavity sequentially and being blown toward the scalp of the user. The above light therapy device can cool the head of the user during the treatment process, improve the user's experience, enable the user to accept treatment for a longer time, and improve the phototherapy effect and the safety of the device.

### TECHNICAL FIELD

The present disclosure relates to a technical field of medical equipment, especially relates to a phototherapy device and a phototherapy apparatus for cerebral functional related diseases.

### BACKGROUND

In recent years, Research studies have found that light can be used to improve cerebral function in the research and treatment of neurological and mental diseases, especially near-infrared light. For example, near-infrared light can be used to treat Alzheimer's disease, depression, autism and other cerebral functional related diseases. Phototherapy apparatus needs to apply light source to transmit transcranial light to the scalp, so as to improve cerebral function. However, in the prior art, the phototherapy apparatus usually provides a relatively low irradiance, which is not sufficient to bring about satisfactory treatment effect, however, adopting a high average irradiance will bring many other problems. For example, the light source of the phototherapy apparatus will generate a lot of heat during operation, resulting in a high temperature of the user's scalp during the phototherapy process, which will bring a strong sense of discomfort to the user, making it difficult for the user to accept a longer treatment time. The existing phototherapy apparatus does not have the design to cool efficiently the user's head, and excessive temperature may even cause scald, which not only affects negatively the phototherapy effect, but also reduces the safety of the apparatus, resulting in poor user experience.

### SUMMARY

In view of the above technical problems in the prior art, the present disclosure provides a phototherapy device and a phototherapy apparatus for cerebral functional related diseases, which can lower efficiently the temperature of the user's head during the treatment process of cerebral functional related diseases, improve the user experience, enable the user to follow a longer-term treatment, and improve the phototherapy effect and the safety of the apparatus.

In the first aspect, the embodiment of the present disclosure provides a phototherapy device for cerebral functional related diseases, including a headgear, an irradiation assembly and a cooled gas supply mechanism. The headgear comprises a housing assembly, inner side of which forms a spaced cavity with scalp of a user and is provided with a first gas inlet. The spaced cavity is constructed to form gas pathway between the first gas inlet and the scalp of the user. The irradiation assembly is mounted to the housing assembly with its light emergent side located to the inner side of the housing assembly. The cooled gas supply mechanism is interconnected to the first gas inlet through the housing assembly and supplies the cooled gas for passing through the first gas inlet and the spaced cavity sequentially and being blown toward the scalp of the user.

In the second aspect, the embodiment of the present disclosure provides a phototherapy apparatus for treatment of Alzheimer's disease, including the phototherapy device of any embodiment of the present disclosure. The phototherapy device includes a headgear, an irradiation assembly and a cooled gas supply mechanism. The headgear comprises a housing assembly, inner side of which forms a spaced cavity with scalp of a user and is provided with a first gas inlet. The spaced cavity is constructed to form gas pathway between the first gas inlet and the scalp of the user. The irradiation assembly is mounted to the housing assembly with its light emergent side located to the inner side of the housing assembly. The cooled gas supply mechanism is interconnected to the first gas inlet through the housing assembly and supplies the cooled gas for passing through the first gas inlet and the spaced cavity sequentially and being blown toward the scalp of the user.

In the third aspect, the embodiment of the present disclosure provides a phototherapy apparatus for treatment of mental disease and mental disorder including depression, including the phototherapy device of any embodiment of the present disclosure. The phototherapy device includes a headgear, an irradiation assembly and a cooled gas supply mechanism. The headgear comprises a housing assembly, inner side of which forms a spaced cavity with scalp of a user and is provided with a first gas inlet. The spaced cavity is constructed to form gas pathway between the first gas inlet and the scalp of the user. The irradiation assembly is mounted to the housing assembly with its light emergent side located to the inner side of the housing assembly. The cooled gas supply mechanism is interconnected to the first gas inlet through the housing assembly and supplies the cooled gas for passing through the first gas inlet and the spaced cavity sequentially and being blown toward the scalp of the user.

Compared with the prior art, the beneficial effect of the embodiments of the present disclosure is as follows. In the phototherapy device of the present disclosure, the cooled gas supply mechanism supplies cooled gas, which is delivered through the hollow cavity of the housing assembly (i.e., through the housing assembly), and then the first gas inlet to the above spaced cavity formed between the housing assembly and the user's scalp. The cooled gas may flow relatively smoothly and performs heat convection in the sufficient spaced cavity, and thus removes the heat from and cools the user's head efficiently. It solves the problem that the heat generated by the irradiation assembly in the prior art causes the user's overheat discomfort on the head (e.g., the scalp). The present disclosure not only effectively improves the user's comfort, improves the phototherapy effect, but also improves the safety of the equipment, so as to avoid scalding the user.

### BRIEF DESCRIPTION OF THE DRAWINGS

In figures that are not necessarily drawn to scale, the same reference numerals may describe similar components in different figures. The same reference signs with suffixes or different suffixes may denote different examples of similar components. The figures generally show various embodiments by way of example rather than limitation, and are used together with the description and the claims to describe the embodiments of the present disclosure. As proper, the same reference sign may be used throughout the drawings to denote the same or similar part. Such embodiments are illustrative, and are not intended to be exhaustive or exclusive embodiments of the present device or method.

FIG. 1 is a sectional view of the phototherapy device according to a first embodiment of the present disclosure;

FIG. 2 is a schematic perspective view of the phototherapy device according to the first embodiment of the present disclosure;

FIG. 3 is a schematic perspective view of the phototherapy device according to the first embodiment of the present disclosure, from another viewing angle different from that as shown in FIG. 2;

FIG. 4 is a schematic diagram of the phototherapy device according to an embodiment of the invention, which is being worn by the patient;

FIG. 5 is the structural schematic diagram of the spiral wind guide structure of the phototherapy device according to the first embodiment of the present disclosure;

FIG. 6 is the structural schematic diagram of the retaining structure of the phototherapy device according to the first embodiment of the present disclosure;

FIG. 7 is an enlarged view of Part A in FIG. 1;

FIG. 8 is a schematic perspective view of the phototherapy device according to a second embodiment of the present disclosure;

FIG. 9 is a schematic perspective view of the phototherapy device according to a third embodiment of the present disclosure;

FIG. 10 is a plan view of the phototherapy device according to the third embodiment of the present disclosure;

FIG. 11 is a structural schematic diagram of the headgear of the phototherapy device according to an embodiment of the present disclosure, which is suspended on the bracket. The components indicated by the reference signs in the figures are as follows:

100—headgear; 1—housing assembly; 11—outer housing; 12—inner housing; 13—spacer; 14—first chamber; 15—second chamber; 16—first gas inlet; 17—second gas inlet; 18—gas extraction port; 19—air flow hole; 110—light transmissive sleeve; 2—cooled gas supply mechanism; 21—gas ventilator pipe; 3—hot gas pumping and discharging mechanism; 31—gas extraction pipe; 4—irradiation assembly; 41—optical radiator; 42—heat sink; 5—spiral wind guide structure; 6—retaining structure; 61—stop plate; 62—gas extraction hole; 7—socket base; 8—support assembly; 81—support bodies; 82—second vent; 9—bracket; 10—branch pipeline assembly; 101—branch pipe

### DETAILED DESCRIPTION

Various solutions and features of the present disclosure are described herein with reference to the accompanying drawings.

It should be understood that various modifications can be made to embodiments of the present disclosure herein. Therefore, the above description should not be regarded as a limitation, but only as an example of an embodiment. Those skilled in the art will conceive of other modifications within the scope and spirit of the present disclosure.

The accompanying drawings, which are included in and constitute a part of the description, show embodiments of the present disclosure, and are used to explain the principles of this disclosure together with the general description of this disclosure given above and the detailed description of the embodiments given below.

These and other features of the present disclosure will become apparent from the following description of preferred forms of embodiments given as non-limiting examples with reference to the accompanying drawings.

It should also be understood that although the present disclosure has been described with reference to some specific examples, those skilled in the art can definitely implement many other equivalent forms of the present disclosure.

When combined with the drawings, the above and other aspects, features and advantages of the present disclosure will become more apparent in view of the following detailed description.

Hereafter, specific embodiments of the present disclosure will be described with reference to the accompanying drawings. However, it should be understood that the embodiments of the invention are only examples of the present disclosure, which can be implemented in a variety of manners. Familiar and/or repetitive functions and structures are not described in detail to avoid unnecessary or superfluous details that obscure the present disclosure. Therefore, the specific structural and functional details of the invention herein are not intended to be limited, but are only used as the basis and representative basis of the claims to teach those skilled in the art to use this disclosure in a diversified manner with any virtually suitable detailed structure.

The description may use the phrases “in one embodiment”, “in another embodiment”, “in yet another embodiment” or “in other (some) embodiments”, which may refer to one or more of the same or different embodiments according to this disclosure.

The embodiment of the present disclosure provides a phototherapy device for cerebral functional related diseases. The phototherapy device is adapted to any high average irradiance (sometimes represented by or referred as power density) phototherapy apparatus, including but not limited to the treatment of Alzheimer's disease, depression, etc., and it is also not limited from the aspects of irradiation parameters such as wavelength and pulse frequency of transcranial light. The “cerebral functional related diseases” in the description are intended to include diseases with abnormalities in at least one aspect, of the macro or micro structure, biochemical conditions and metabolic conditions of the brain, such as but not limited to Alzheimer's disease, depression, etc.

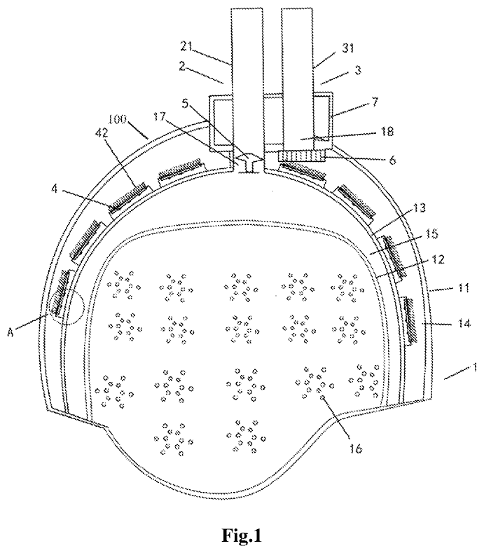

As shown in FIGS. 1 to 3, the phototherapy device includes a headgear 100, an irradiation assembly 4, and a cooled gas supply mechanism 2. The headgear 100 includes a housing assembly 1. A spaced cavity is formed between the inner side of the housing assembly 1 and the user's scalp, and the inner side of the housing assembly 1 is provided with a first gas inlet 16, wherein, the spaced cavity is constructed to form gas pathway between the first gas inlet 16 and the user's scalp. The irradiation assembly 4 is mounted to the housing assembly 1, and its light emergent side is located on the inner side of the housing assembly 1. The cooled gas supply mechanism 2 is interconnected to the first gas inlet 16 through the housing assembly 1, and supplies the cooled gas for passing through the first gas inlet 16 and the spaced cavity sequentially and being blown toward the scalp of the user. Throughout the description, the term “interconnect” means providing fluid interconnections, i.e., “A is interconnected to B” means fluid may be delivered from one of A and B to another. As an example, such interconnection may be implemented by an intermediate pipe coupling A to B, or also may be implemented by interfacing a hole in a hollow A with a hole in a hollow B, etc. As an example, the cooled gas supply mechanism 2 supplies cooled gas, which is delivered through at least part of the hollow cavity of the housing assembly 1 (an example for implementing “through the housing assembly 1”), and then the first gas inlet 16 to the above spaced cavity formed between the housing assembly 1 and the user's scalp. The cooled gas flows relatively smoothly and performs heat convection in the sufficient spaced cavity, and thus removes the heat from and cools the user's head efficiently.

Specifically, the spaced cavity can correspond to any part, including but not limited to the top part, the peripheral part, etc., of the user's head. Preferably, the spaced cavity is provided corresponding to both the top part together with the peripheral part of the user's head, so that each part of the user's head can feel the gas entering the spaced cavity, thus improving the comfort. It should be noted that the inside in this disclosure refers to the side of each component close to the user's head. Furthermore, the spaced cavity has sufficient volume, which allows the user's head (or its substantial portion) to be contained in it in a loose manner rather than closely matched, so as to provide sufficient heat exchange and convection between the cooled gas and the scalp, enabling the heat near the scalp to be quickly removed at a comfortable air flow speed.

Specifically, the above spaced cavity can be formed by providing components capable of supporting on the inner side of the housing assembly 1, or by the shape of the housing assembly 1 itself.

Specifically, the irradiation assemblies 4 can be multiple, and each irradiation assembly 4 is used to transmit transcranial light to the user's head, so that more transcranial light can be delivered to the cerebral cortex, thereby improving the therapeutic effect on brain diseases. In some embodiments, the above transcranial light may be near-infrared light.

Specifically, the first gas inlet 16 is used to guide the cooled gas introduced by the cooled gas supply mechanism 2 into the spaced cavity to achieve cooling. The cooled gas supply mechanism 2 can be independently provided outside the headgear 100 or on the housing assembly 1 of the headgear 100. As long as it can provide the first gas inlet 16 with the gas that can achieve cooling. The temperature of the gas can be adjusted according to the actual needs (e.g., of the individual users), such as the temperature in the spaced cavity, the temperature of the scalp, the amount of sweat on the scalp. In this manner, the temperature in the spaced cavity can be decreased to provide a comfortable environment for the individual user's head.

In some optional embodiments, the above-mentioned cooled gas supply mechanism 2 can be connected with (or include) a machine capable of producing cooled gas, such as a refrigerator. The refrigerator can be understood as a compressor. After producing cooled gas, the refrigerator transmits the cooled gas to the cooled gas supply mechanism 2, which then transmits the cooled gas to the spaced cavity. Particularly, the cooled gas is discharged to the scalp through the first gas inlet 16 interconnected with the spaced cavity, so as to achieve the effect of cooling the scalp. The comfort degree of the user when wearing the headcap 100 for phototherapy is improved, so that the temperature near the user's scalp can be controlled stably between 18° C. and 43° C. Preferably, the temperature near the user's scalp is ensured to be not higher than 40° C. Because such controlled temperature is close to the body temperature, even users who are very sensitive to temperature and have strict tolerance will feel comfortable, and will not cause thermal damage, so that they can accept and follow continuous and persistent treatment. With such an cooled gas supply mechanism 2 in combination with the loosely designed housing assembly 1 described previously, users are more willing to accept and follow persistent light irradiation treatment, and a single irradiation fraction (irradiation per time) can also last for a longer time (for example, it can be as long as 20 minutes, or even 30 minutes) and fractionated dose may be increased properly, so as to further improve the treatment effect. The longer the fraction duration period is, the higher the heat production near the scalp is. Besides, the higher the average irradiance is, the higher the heat production near the scalp is.

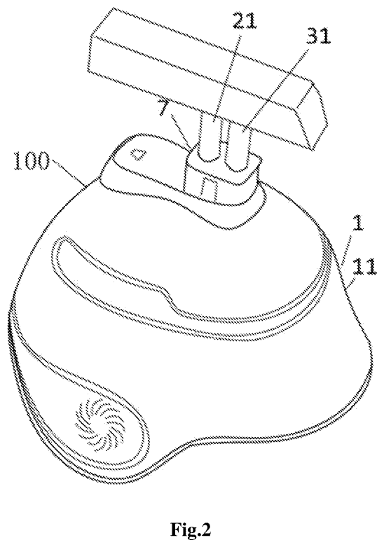

In some optional embodiments, as shown in FIGS. 1 and 2, the above cooled gas supply mechanism 2 can be connected to the top of the housing assembly 1 and interconnected to the first gas inlet 16 through (e.g., the hollow cavity of) the housing assembly 1. Wherein, the cooled gas supply mechanism 2 can pass through the housing assembly 1 by means of a structure such as a pipe or a connector.

The present disclosure supplies cooled gas to the above spaced cavity formed between the housing assembly 1 and the user's scalp through the first gas inlet 16 by the cooled gas supply mechanism 2, thus realizing the cooling of the user's head through the cooled gas introduced into the spaced cavity. It solves the problem that the heat generated by the irradiation assembly 4 in the prior art causes the user's head discomfort. The present disclosure not only effectively improves the user's comfort, improves the phototherapy effect, but also improves the safety of the equipment, so as to avoid scalding the user.

In some embodiments, the cooled gas is a kind of gas with adjustable temperature obtained by refrigeration of a compressor. In this way, the temperature and volume of the cooled gas produced can be adjusted by controlling the operation state of the compressor, so as to improve the cooling experience of the head of the user.

In some embodiments, as shown in FIGS. 8 to 10, the headgear 100 further includes a support assembly 8 provided to the inner side of the housing assembly 1, and the support assembly 8 is constructed to maintain the spaced cavity between the housing assembly 1 and the user's scalp.

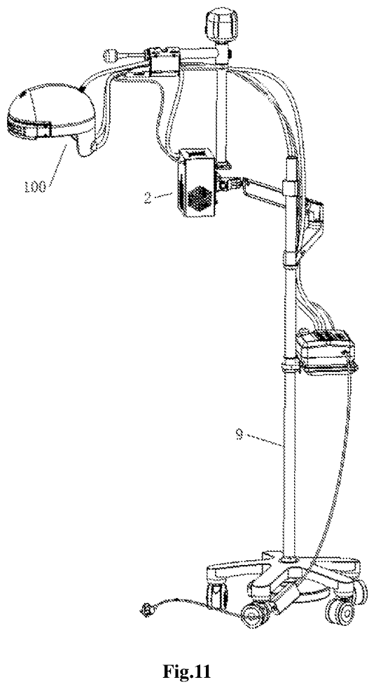

Specifically, the above spaced cavity can be understood as a space between the headgear 100 and the user's scalp after the user wears the headgear 100. It should be noted that the spacing between the housing assembly 1 and the user's scalp can be made by the support function of the support assembly 8, or the spacing can be generated by other manners. For example, as shown in FIG. 11, a suspension component can be provided on the headgear 100. During using the phototherapy device, the headgear 100 can be suspended on the bracket 9 so that the headgear 100 does not contact the scalp to generate the spacing. That is, the spacing is not generated by the support function of the support component 8, and the support component 8 may not contact against with the scalp of the user, or may not completely contact against the scalp of the user. The support component 8 may be used to maintain and ensure a sufficient spaced cavity between the housing assembly 1 and the scalp even when the spacing between the headgear 100 and the scalp changes. As an example, when the distance between the housing assembly 1 and the scalp decreases due to the movement of the user's head, the support assembly 8 will support against the scalp to maintain a spaced cavity formed consistently between the housing assembly 1 and the scalp.

It can be understood that the support assembly 8 can be provided on the inside of the housing assembly 1 at any position, such as positions corresponding to the top part, peripheral part, etc., of the head, as long as a spaced cavity can be formed between the housing assembly 1 and the user's scalp.

Particularly, the support assembly 8 can include components such as an air bag, which can be filled with media through an air pump. The air bag may adapt to the head shape of the user, thereby improving the wearing comfort. The air pump can be independently provided outside the headgear 100 or on the housing assembly 1, which is not specifically defined in this application.

Particularly, a flexible layer may be provided to the above support assembly 8 on its scalp side, so as to further improve the user's experience when it is against the scalp.

Particularly, the above support assembly 8 can be provided around the user's head.

In some embodiments, the housing assembly 1 includes an outer housing 11, a euphotic (translucent or transparent) inner housing 12, and a spacer 13. A chamber is formed between the outer housing 11 and the inner housing 12. The spacer 13 is used to divide the chamber into a first chamber 14 as outer layer and a second chamber 15 as inner layer. The irradiation assembly 4 is provided within the first chamber 14, and the first gas inlet 16 is opened onto the inner housing 12. The cooled gas supply mechanism 2 is interconnected to the first gas inlet 16 through the second chamber 15, for the cooled gas to be blown toward the scalp of the user by passing through the second chamber 15 and the first gas inlet 16 sequentially.

Particularly, the above inner housing 12 can be made of light transmissive material, which is transmissible for the transcranial light. As an example, for the transcranial light, the material of inner housing 12 may be euphotic.

Particularly, the above outer housing 11 can be understood as a housing located at the outer layer away from the head when the headgear 100 is worn, and the inner housing 12 can be understood as a housing located at the inner layer close to the head when the headgear 100 is worn, and the shapes of both the outer housing 11 and the inner housing 12 can be substantially headgear shaped with arched surface. The spacer 13 is located between the outer housing 11 and the inner housing 12, and the shape of the spacer 13 can correspond to the shapes of both the outer housing 11 and the inner housing 12. For example, the shape of the spacer 13 may also be headgear shaped.

Particularly, the outer housing 11 and the spacer 13 together encases and forms the above first chamber 14, while the inner housing 12 and the spacer 13 together encases and forms the above second chamber 15. The irradiation assembly 4 is provided in the first chamber 14, which can keep the irradiation assembly 4 away from the head to a certain extent, thereby reducing the impact of the heat generated by the operation of the irradiation assembly 4 on the head and ensuring a certain degree of safety. In order to ensure that the transcranial light emitted by the irradiation assembly 4 can directly irradiate the scalp, the inner housing 12 and the spacer 13 are made of euphotic materials to reduce the loss of transcranial light and guide the transcranial light to the scalp as much as possible.

In some optional embodiments, the first gas inlet 16 opened onto the inner housing 12 can be used not only to blow the cooled gas in the second chamber 15 to the scalp, but also to transmit light, i.e., the light emitted by the irradiation assembly 4 is emitted to the scalp through the first gas inlet 16. In addition, the size of the above first gas inlet 16 can be determined according to the air volume and wind velocity blowing to the scalp, so as to increase the comfort of the cooled gas discharged from the first gas inlet 16 blowing to the scalp by means of the appropriate sizing of the first gas inlet 16, and improve the user's feeling and experience, especially for some users with poor tolerance, who may also accept and follow phototherapy meanwhile enjoying the improved comfort.

In some optional embodiments, as shown in FIG. 4, after the user wears the above phototherapy device, a spaced cavity is formed between the inner housing 12 of the headgear 100 and the user's scalp. That is, the user's head does not cling to the inner surface of the inner housing 12. The cooled gas discharged from the first gas inlet 16 enters the spaced cavity to provide a more comfortable phototherapy environment for the user. The first gas inlet 16 is prevented from being blocked by the user's head, thus ensuring that the cooled gas can be discharged smoothly. Particularly, the above housing assembly 1 has a fixed structure and size, which can accommodate the head of the user in a loose manner. Particularly, the structure of the housing assembly 1 leaves out a certain margin, so that when the user rotates his/her head within the preset angle range or moves up and down within the preset distance range, the housing assembly 1 can still cover the region of interest (ROI), so that the light emitted by the irradiation assembly 4 penetrates through the user's skull and passes into each ROI if necessary. Furthermore, the loose and open design of the housing assembly 1 can adopt a fixed structure and size for users who have individual differences in head shape and size to a certain extent, and thus it may not be necessary to customize the housing assembly 1 that strictly matches the head shape and size for individual user. Thus, the housing assembly 1 can be manufactured in a standardized manner at a lower cost, and the headgear 100 can adapt to a wider user population. This reduces the purchase and maintenance costs of the facilities in medical places such as hospitals and communities where the phototherapy devices are used. Particularly, the so-called fixed structure and size means that the housing assembly 1 may not be provided with movable components, and may even be molded into one piece, thus increasing the service life of the housing assembly 1 and simplifying the structure of the housing assembly 1.

In some embodiments, the phototherapy device further includes a hot gas pumping and discharging mechanism 3, which is interconnected to the first chamber 14 and constructed to extract the hot gas heated by the heat generated by the irradiation assembly 4 and discharge it outside the housing assembly 1.

In some optional embodiments, the hot gas pumping and discharging mechanism 3 can be connected with a machine capable of pumping gas, such as an exhaust fan, which sucks out the hot gas in the first chamber 14 through the hot gas pumping and discharging mechanism 3. Of course, in order to ensure the stability of the air pressure in the first chamber 14, the above first chamber 14 can be interconnected with the outside world, so that the outside gas (such as the atmosphere) can enter the first chamber 14 in time when the hot gas is sucked out. Thus, the air pressure in the first chamber 14 is maintained at atmospheric pressure.

In some optional embodiments, in the above first chamber 14, other elements or components that will generate heat during operation, such as sensors, mainboards, etc., may be further provided. The hot gas pumping and discharging mechanism 3 can not only extract the hot gas formed by the heat generated by the irradiation assembly 4, but also extract the hot gas formed by the heat generated by other elements or components in the first chamber 14 during operation.

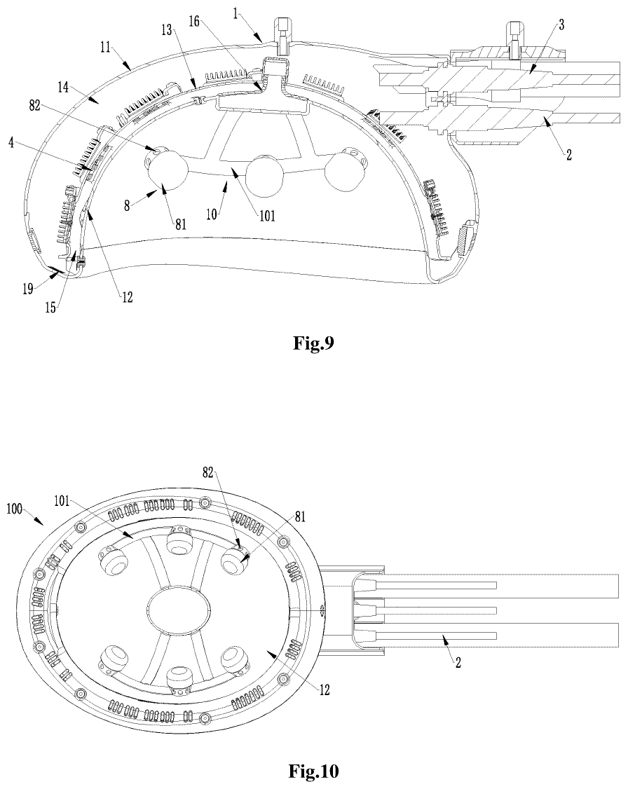

In some embodiments, as shown in FIG. 9, the headgear 100 also includes a branch pipeline assembly 10, which is provided to the inner side of the housing assembly 1 and is interconnected to the first gas inlet 16. A first vent is opened onto the branch pipeline assembly 10, and the branch pipeline assembly 10 includes a plurality of branch pipes 101 paved in the spaced cavity.

Particularly, the gas supplied from the first gas inlet 16 enters the branch pipeline assembly 10 and is discharged to the spaced cavity through the first vent. The branch pipeline assembly 10 can reduce the pressure of the gas entering it, and is used to discharge the gas evenly and dispersedly to the spaced cavity, so as to prevent the user from discomfort caused by excessively fast flow rate of the cooled gas.

Particularly, the first vent can be provided onto the branch pipe 101 to directly discharge the gas through the branch pipe 101.

Particularly, the side of the branch pipe 101 toward the scalp can form a support surface for supporting the head, thereby assisting the support assembly 8 in providing a more stable support function.

Particularly, the above branch pipeline assembly 10 and the support assembly 8 can be provided on the inner side of the inner housing 12. In order to avoid blocking the light path, both the branch pipeline assembly 10 and the support assembly 8 can be made of light transmissive materials.

In some embodiments, as shown in FIG. 9, the support assembly 8 includes a plurality of support bodies 81 provided on the inner housing 12. The support body 81 is opened with a second vent 82, and is interconnected to the branch pipe 101, so that the gas in the branch pipe 101 is discharged to the spaced cavity through the second vent 82.

In some optional embodiments, the support body 81 and the branch pipe 101 of the branch pipeline assembly 10 are provided independently.

Particularly, the second vent 82 can form part or all of the above first vent, that is, the first vent of the branch pipeline assembly 10 can be constructed as the second vent 82 of the support body 81. At this time, there is no need to open additional vent hole on each branch pipe 101, and the gas can be discharged through the second vent 82 on (in) the support body 81.

Particularly, the above support body 81 can be distributed on the branch pipe 101, the support body 81 can be connected to the branch pipe 101 in a detachable manner, or the support body 81 can be integrally formed onto the branch pipe 101, making the structural design of the above headgear 100 more compact and the layout more reasonable.

Particularly, the opening orientation of the second vent 82 located on (in) the support body 81 can be inclined with respect to scalp-oriented direction, so that the low temperature gas located on the top of the head can naturally gradually diffuse to the entire spaced cavity from top to bottom, so as to improve the user's comfort.

In some embodiments, the opening orientation of the first vent is inclined with respect to the scalp-oriented direction, so as to avoid cooled gas blowing directly to the scalp and causing discomfort to the user.

In some embodiments, as shown in FIG. 1, the first chamber 14 and the second chamber 15 are independent from each other. That is, there is no gas flow between the first chamber 14 and the second chamber 15. In this way, the hot gas in the first chamber 14 can be prevented from spreading to the second chamber 15, causing more heat to dissipate to the user's head causing discomfort, and the cooled gas for cooling the user's head can also be prevented from entering the first chamber 14, affecting the cooling effect on the user's head.

Particularly, the lower end of the spacer 13 can be sealed with the outer housing 11 and/or the inner housing 12, so that the first chamber 14 and the second chamber 15 on opposite sides of the spacer 13 can be independent from each other.

Particularly, the above spacer 13 can be made of materials with poor thermal conductivity to prevent more heat in the first chamber 14 from being transmitted to the spacer 13, avoiding the temperature of the second chamber 15 to rise accordingly.

In some embodiments, the first chamber 14 and the second chamber 15 are not completely independent of each other. Specifically, the first chamber 14 and the second chamber 15 can be interconnected through tiny lumens. In this case, a small part of the cooled gas in the second chamber 15 may enter the first chamber 14 through tiny lumens, thereby assisting the cooling of the components in the first chamber 14.

In some embodiments, as shown in FIGS. 8 and 9, an air flow hole 19 is opened onto the cavity of the first chamber 14.

Particularly, in the above first chamber 14, a plurality of elements or components for supporting each function of the headgear 100, such as a circuit board, a connecting wire, etc., may be further provided. Several such components will generate heat during operation, and the above air flow hole 19 can take away part of the heat in the first chamber 14, so as to achieve the purpose of cooling the first chamber 14. In one embodiment, a cooling assembly, such as a fan, can be provided in the first chamber 14. The rotation of the fan drives the gas flow in the first chamber 14 to take away the heat in the first chamber 14, thereby reducing the temperature of each element or component in the first chamber 14.

Particularly, the gas can enter the first chamber 14 through the air flow hole 19, and/or the gas in the first chamber 14 can also be discharged through the air flow hole 19. The above air flow hole 19 can be opened onto the bottom or side of the outer housing 11. For example, the air flow hole 19 shown in FIG. 8 is located on the side wall of the outer housing 11, and the air flow hole 19 shown in FIG. 9 is located on the bottom of the outer housing 11. The above arrangements of the air flow hole 19 serve as examples. This application does not limit the specific location of the air flow hole 19, as long as the first chamber 14 can be interconnected to the external environment via the air flow hole 19.

Particularly, a light shielding layer can be provided near the air flow hole 19 between the inner housing 12 and the spacer 13, so as to prevent the light emitted by the irradiation assembly 4 from being emitted out through the air flow hole 19 to cause damage to the human eyes. The above light shielding layer can be a black light shielding plate, a light reflecting plate and other structures.

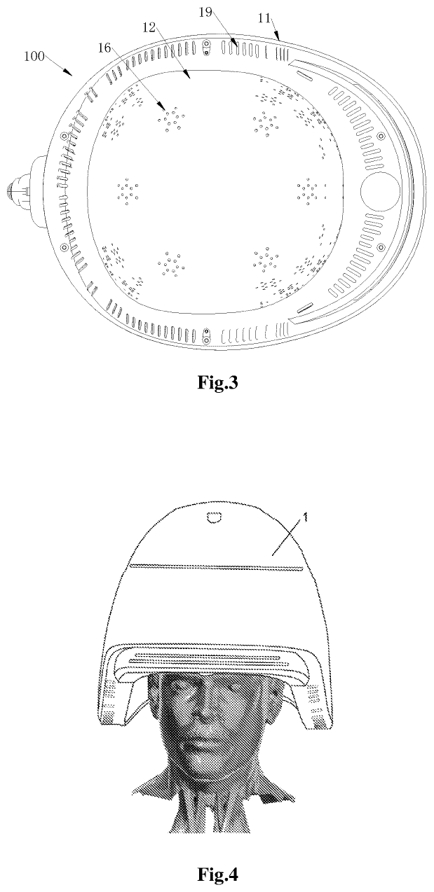

In some embodiments, as shown in FIGS. 1 and 3, the top portion of the spacer 13 is opened with a second gas inlet 17, which is constructed to introduce the cooled gas from the cooled gas supply mechanism 2, and the outer top surface of the inner housing 12 is formed with a wind guide surface provided corresponding to the second gas inlet 17, so as to guide the cooled gas introduced via the second gas inlet 17 to the periphery to realize heat dissipation from the whole head.

In some embodiments, the wind guide surface can be constructed as a concave, arch or any other structure that can make the cooled gas diverge around. The top portion of the inner housing 12 is arch-shaped with a curvature less than a preset curvature, so that the introduced cooled gas diffuses gently to the periphery under the action of the arch-shaped top portion of the inner housing 12.

In some optional embodiments, the curvature of the arch can be as small as possible, so that a large amount of cooled gas entering the second gas inlet 17 can be directly blown to the top portion of the inner housing 12, and the flow rate of cooled gas can be reduced under the function of the top portion of the inner housing 12, and then the cooled gas can diffuse to the periphery of the top portion of the inner housing 12, so that the cooled gas can be discharged from each first gas inlet 16 smoothly and evenly, improving user comfort.

Particularly, the outer housing 11 can be opened with a mounting port corresponding to the second gas inlet 17, through which the cooled gas supply mechanism 2 is connected to the second gas inlet 17.

Particularly, the above preset curvature can be set according to experience, wind velocity of the cooled gas, etc., which is not particularly limited in this application.

In some embodiments, as shown in FIGS. 1 and 3, at least the central part of the top portion of the inner housing 12 is flatten-shaped.

In some optional embodiments, the flatten-shaped plane at the top portion of the inner housing 12 is provided corresponding to the second gas inlet 17 in the up-down direction, so that the cooled gas entering the second gas inlet 17 can flow directly to the direction of the flatten-shaped plane.

In some embodiments, as shown in FIGS. 1 and 5, the second gas inlet 17 is provided with a spiral wind guide structure 5 to guide the flow direction of the introduced cooled gas, so that the cooled gas can be blown to the periphery and further improve the uniform cooling effect.

In some optional embodiments, the above spiral wind guide structure 5 is detachably installed at the second gas inlet 17. Particularly, the spiral wind guide structure 5 can include a base plate and a spiral wind guide vane provided on the base plate. The base plate can be lapped (or bonded in other manners) onto the second gas inlet 17, and the base plate is provided with a through slot corresponding to the spiral wind guide vane, so that the cooled gas can be blown from the through slot to the spiral wind guide vane, and then delivered into the second chamber 15 after spirally guided by the spiral wind guide vane.

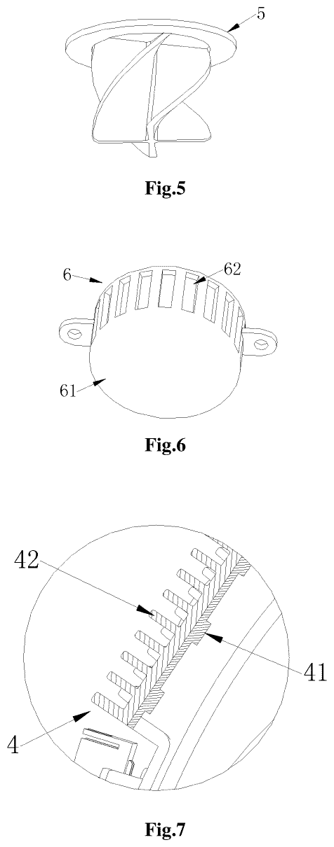

In some embodiments, as shown in FIGS. 1 and 6, a gas extraction port 18 is opened in the outer housing 11, which is interconnected to the hot gas pumping and discharging mechanism 3, the gas extraction port 18 is provided with a retaining structure 6. The retaining structure 6 includes a stop plate 61 and a plurality of gas extraction holes 62 interconnected to the first chamber 14, and the stop plate 61 is provided corresponding to the gas extraction port 18 in the up-down direction.

In this manner, it can shield the first chamber 14 and the gas extraction port 18, and prevent foreign matters from falling into the first chamber 14 via the gas extraction port 18. It also prevents the operator from putting his/her hand into the first chamber 14 through the gas extraction port 18 by mistaken operation, thus improving the safety of the apparatus.

In some optional embodiments, the above retaining structure 6 can have a cylindrical body, the stop plate 61 is configured as the bottom of the cylindrical body, each gas extraction hole 62 is located on the cylinder wall of the cylindrical body, and the gas in the first chamber 14 is extracted through the gas extraction hole 62 at the cylinder wall.

In some optional embodiments, the opposite sides of the retaining structure 6 can be extended to form a connection portion, by means of which the retaining structure 6 is installed at the gas extraction port 18.

In some optional embodiments, the gas extraction port 18 is provided on the top portion of the outer housing 11, and the gas extraction port 18 can be provided close to the second gas inlet 17, which makes the structural design of the headgear 100 more compact and the layout more reasonable.

In some embodiments, as shown in FIG. 6, a plurality of the gas extraction holes 62 are distributed in an annular shape around the stop plate 61, so that the gas in the first chamber 14 can be evenly sucked into the hot gas pumping and discharging mechanism 3 via the gas extraction holes 62 distributed in an annular shape.

Particularly, the above gas extraction holes 62 can be uniformly distributed around the stop plate 61.

In some embodiments, as shown in FIGS. 1 and 3, a plurality of irradiation assemblies 4 are provided in array, with the array of the irradiation assemblies 4 provided to the outer side of the spacer 13. A plurality of first gas inlets 16 are provided in groups, with each group of the first gas inlets 16 corresponding to each individual irradiation assembly 4 respectively. In some embodiments, the array of the irradiation assemblies 4 is not necessarily provided on the spacer 13.

In some optional embodiments, the array mode of the above irradiation assemblies 4 can be set according to the treatment scheme. For example, when the above phototherapy device is used to treat depression, it can target the forehead and left and right temporal lobes of the human brain. Therefore, the irradiation assemblies 4 can be provided in the first chamber 14 at positions corresponding to the forehead and left and right temporal lobes of the head, to perform phototherapy with respect to the forehead and left and right temporal lobes of the user. As another example, when the above phototherapy device is used to treat Alzheimer's disease, it can treat the whole brain of a patient. Therefore, the irradiation assemblies 4 can be added in the first chamber 14 at the positions corresponding to the whole brain, to perform phototherapy with respect to the whole brain of the user. In this manner, the array mode of the irradiation assemblies 4 can be adaptively selected based on different symptoms for targeted treatment.

Further, after determining the array mode of the irradiation assemblies 4, the arrangement mode of the first gas inlet 16 of each group can be determined according to the array mode of the irradiation assembly 4, that is, the vent position of the cooled gas can be adjusted, so that the air volume and wind velocity of the cooled gas discharged from the first gas inlet 16 can be more suitable for users.

In some optional embodiments, the above irradiation assemblies 4 are provided in one-to-one correspondence with the groups of first gas inlets 16, that is, one irradiation assembly 4 corresponds to a group of first gas inlets 16.

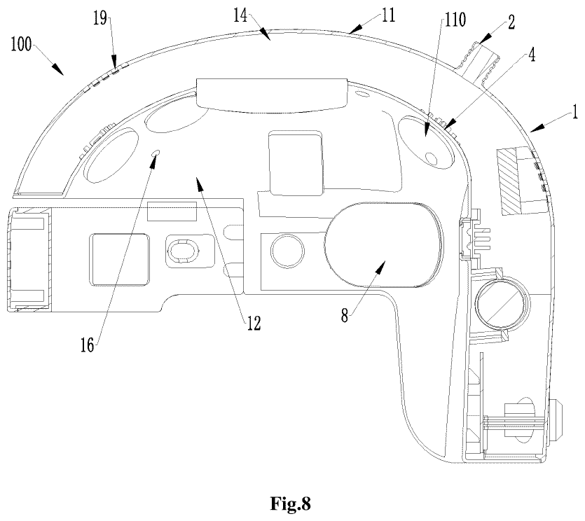

In some embodiments, as shown in FIG. 7, the irradiation assembly 4 includes a lamp panel and a plurality of optical radiators 41 provided onto its first plate, and the plurality of optical radiators 41 are staggered from at least part of the group of the first gas inlets 16 corresponding to the lamp panel where the plurality of optical radiators are located, so as to avoid that the temperature of the user's scalp is too high after the transcranial light directly irradiates on the user's scalp. Through the staggered arrangement, the wind velocity can also be controlled to make the user comfortable, preventing wind velocity adjacent to the scalp from becoming too fast, so as to improve the user experience.

Particularly, except for the one or more first gas inlets 16 staggered with the optical radiators 41, the other first gas inlets 16 in the group can be set corresponding to the optical radiators 41, so that the transcranial light emitted by the optical radiator 41 can directly irradiate on the scalp in the corresponding region as proper, bringing better phototherapy effect.

Particularly, the optical radiator 41 can be configured as a near-infrared light-emitting diode.

In some embodiments, the number of first gas inlets 16 in each group is greater than the number of optical radiators 41 on the corresponding irradiation assembly 4. In this manner, the uniform discharge effect of cooled gas can be improved by increasing the number of the first gas inlets 16, so as to achieve a better cooling effect for the scalp.

As an example, the number of first gas inlets 16 included in a group of first gas inlets 16 can be 11 to 15, and the number of optical radiators 41 on a corresponding irradiation assembly 4 can be 8 to 10.

In some embodiments, the inner housing 12 has an area for opening the groups of the first gas inlets 16, which comprises a first area covering the corresponding lamp panel and a second area surrounding the first area. Particularly, the first area covers the corresponding irradiation assembly 4, and the second area surrounds the first area to increase the cooled gas blowing area of the first gas inlet 16 and improve the comfort of the scalp during phototherapy.

In some embodiments, the hole diameter of the group of first gas inlets 16 located below is larger than that of the group of first gas inlets 16 located above it. Wherein, the group of first gas inlets 16 located below can be understood as the first gas inlets 16 relatively far away from the corresponding overhead area on the inner housing 12, and the group of first gas inlets 16 located above can be understood as the first gas inlet 16 relatively close to the corresponding overhead area on the inner housing 12. In this manner, the cooled gas can be discharged more evenly through the first gas inlets 16 to improve the cooling effect and prevent the cooled gas from diffusing more from the top portion, affecting the cooling effect at other locations.

In some embodiments, as shown in FIGS. 1 and 3, the first gas inlets 16 of each group radiate from the center to the periphery radially. In this manner, the cooled gas can flow to the scalp dispersedly and evenly remove and dissipate heat for the scalp.

In some embodiments, as shown in FIGS. 1 and 7, the second plate of the irradiation assembly 4 is provided with a heat sink 42, which extends in the direction away from the spacer 13.

Particularly, the above heat sinks 42 comprises a plurality of sheets provided at intervals, and the heat generated by the operation of the irradiation assembly 4 can be directed to the heat sink 42 and discharged to the first chamber 14 through the intervals between the sheets.

Particularly, the above heat sinks 42 can be made of materials with good thermal conductivity, such as aluminum, copper, etc.

In some embodiments, as shown in FIGS. 1 and 2, the phototherapy device also includes a socket base 7, the cooled gas supply mechanism 2 includes a gas ventilator pipe 21 while the hot gas pumping and discharging mechanism 3 comprises a gas extraction pipe 31, wherein the socket base 7 connects to one end of the gas ventilator pipe 21 and also connects to one end of the gas extraction pipe 31, and is mounted to the outer housing 11 in a detachable manner. In this manner, the gas ventilator pipe 21 and the gas extraction pipe 31 can be connected to or disconnected from the outer housing 11 together through the operation of plugging/pulling the socket base 7 into/off the outer housing 11. The operation is simple and convenient.

In some optional embodiments, the outer housing 11 can be provided with a mounting base corresponding to the second gas inlet 17 and the gas extraction port 18. The socket base 7 can be connected to the mounting base in a detachable manner. After the socket base 7 is installed on the mounting base, the gas ventilator pipe 21 can be interfaced and interconnected to the second gas inlet 17 and the gas extraction pipe 31 can be interfaced and interconnected to the gas extraction port 18.

In some embodiments, the sum of the sectional areas of the plurality of first gas inlets 16 is 2 to 4 times, preferably 3 to 4 times, as large as (larger than) the sectional area of the second gas inlet 17. In this manner, the cooled gas entering via the second gas inlet 17 can be evenly and gently distributed to the scalp through the first gas inlets 16.

In some embodiments, the diameter of the first gas inlet 16 ranges from 1.5 mm to 3 mm.

Preferably, the diameter of the first gas inlet 16 is 2 mm or 2.5 mm.

As an example, the group of first gas inlets 16 located below can each have the hole diameter of about 2.5 mm, and the group of first gas inlets 16 located above can each have the hole diameter of about 2 mm, so that the uniform cooling effect can be better achieved.

In some embodiments, the irradiation assemblies 4 are configured to emit near-infrared light with an average irradiance greater than 40 mW/cm² to the user's head. Under the action of at least the cooled gas supply mechanism 2, the temperature adjacent to the user's scalp is controlled within the range from 23° C. (degrees Celsius) to 40° C. It can be understood that the term “average irradiance” used in this application represents the magnitude of energy of light irradiation on a unit area per unit time.

In some embodiments, the array of the irradiation assemblies 4 are configured to transmit near-infrared light with an average irradiance of 50 mW/cm² to 150 mW/cm² to the user's head, so that the temperature adjacent to the user's scalp is controlled within the range from 23° C. to 40° C. under the action of the cooled gas supply mechanism 2 and the hot gas pumping and discharging mechanism 3.

In some embodiments, the gas pathway formed by the spaced cavity enables the wind velocity adjacent to the user's scalp to be maintained within the range from 0.5 m/s to 3.5 m/s. In this manner, the cooled gas can be gently blown to the head of the user and will not collide directly at the head, so as to avoid discomfort to the user while cooling down.

In some embodiments, the temperature of the cooled gas is tuned, so that the temperature adjacent to the scalp of the user is maintained within the range from 23 degrees Celsius to 40 degrees Celsius in case that the wind velocity adjacent to the scalp of the user is maintained within the range from 0.5 m/s to 3.5 m/s.

In some embodiments, the inner side of the bottom of the housing assembly 1 can be provided with a light shielding part circumferentially, and the light shielding part is used to stop the light emitted by the irradiation assembly 4 from emitting out from the lower part of the headgear 100. The light shielding part is used to prevent the transcranial light from emitting out from the bottom of the housing assembly 1 and causing harm to the human eyes.

In some embodiments, the light shielding part is constructed as an air permeable structure, so that the gas in the spaced cavity can diffuse to the external environment through the air permeable structure.

Particularly, the light shielding part can be constructed as a black sponge with air holes, a black flexible cloth with air permeability, etc., so that the light shielding part can not only maintain a certain degree of comfort, but also have the effect of shading and heat dissipation. Optionally, the shielding structure can also be constructed as a black soft rubber pad (such as silicone pad), a light shielding plate, etc., on which smaller air holes is opened. A reflective structure, such as a mirror, a reflective film, etc., can be provided right above the air holes. The air flow in the spaced cavity diffuses from the air holes to the external environment through the channel on the side of the reflective structure.

In some embodiments, as shown in FIG. 8, the irradiation assembly 4 can be provided with a light transmissive sleeve 110, which is used to prevent the irradiation assembly 4 from being directly exposed to the external environment, so as to improve the safety of the product.

The embodiment of the present disclosure also provides a phototherapy apparatus for treatment of Alzheimer's disease, including the phototherapy device according to any embodiment of present disclosure.

Alzheimer's disease (AD) is more common in the elderly over 65 years old. The onset of AD is slow or insidious, and it worsens over time. Its main performances comprise cognitive function decline, language dysfunction, emotional instability, mental symptoms and behavioral disorders, and gradual decline of daily living ability, which eventually leads to loss of physical function and death. In addition, AD patients have lower sensitivity to temperature and pain, that is to say, AD patients may experience greater pain and potential greater tissue or organ damage before discover and report of injuries. In addition, the elderly over 65 years old account for a large proportion of AD patients, and the elderly are more afraid of cold than the young. Therefore, when using a higher average irradiance or even higher total power to treat AD patients, it is particularly necessary to fully consider the characteristics of AD patient groups' sensitivity to temperature and pain, and provide them with a more comfortable treatment environment that will not cause pain or even heat damage, which will contribute to extend the treatment time, so as to achieve better treatment effect.

The phototherapy apparatus of any embodiment of present disclosure can use near-infrared light to treat AD. And it can supply cooled gas to the spaced cavity, which is formed between the housing assembly 1 and the user's scalp, through the first gas inlet 16 by the cooled gas supply mechanism 2, thus realizing the cooling of the user's head through the cooled gas supplied to the spaced cavity. It solves the problem that the heat generated by the irradiation assembly 4 in the prior art causes the user's head discomfort. The present disclosure not only effectively improves the user's comfort and the phototherapy effect, but also improves the safety of the equipment, so as to avoid scalding the user.

For the Alzheimer's disease, the inventor found through simulation experiments and clinical experiments that the array of the irradiation assemblies 4 of this application is configured to emit near-infrared light with an average irradiance greater than 40 mW/cm² to the head of the user. For elderly AD patients, it can ensure that enough light energy is delivered to the brain tissue and ensure a good treatment effect. Preferably, the average irradiance used can be 40 mW/cm²-150 mW/cm², and the central wavelength of the near-infrared light emitted can be 800-820 nm, for example, 810 nm. For some specific encephalic regions (brain regions), such as the frontal lobe and temporal lobe, the average irradiance of near-infrared light can be more than 50 mW/cm² and up to 150 mW/cm², preferably more than 70 mW/cm².

The housing assembly 1 of the phototherapy device for treating Alzheimer's disease accommodates the head of the patient in a loose manner, so that during the treatment, the head can rotate within a preset angle range and move up and down within a preset distance range. Different from the head shape adapted to the patient, a few gaps at cm level are reserved between the housing assembly 1 and the patient's head, preferably 1-2 cm, so that the patient can move his head within the preset angle range and the preset distance range according to his own wishes. This open design of housing assembly 1 has no sense of constraint on the patient's head. It is especially friendly to the elderly who are restless, anxious, resistant or even afraid of confined or crowded spaces, which can significantly improve the treatment compliance of AD patients. Particularly, the phototherapy device can be used to treat patients suffering from AD and having psychological disorders in confined or crowded spaces. This kind of psychological disorders in confined or crowded spaces can be caused by the patient's age or mental diseases other than AD, or it can be caused by suffering from AD. This design of the housing assembly 1 can also be widely applied to the behavioral characteristics of patients in different courses of AD. For example, for early AD patients, their judgment ability is reduced and they are often suspicious and will be provoked. The wearing of the housing assembly 1 with full freedom and openness is easy to be accepted by patients and not easy to provoke them, so that patients can cooperate with the continuous phototherapy. For another example, for AD patients in the middle stage, their emotions fluctuate violently, they are irritable and restless, and they often walk around consistently. The heads of some patients may shake frequently and slightly without consciousness. This open housing assembly 1 allows the patient's head to shake slightly and unconsciously without causing the housing assembly 1 itself to vibrate with the head. Therefore, it is unnecessary to forcibly stop this small shaking, which increases the comfort of the patient and reduces the workload of the medical staff. At the same time, it can also prevent the shaking of the patient's head from being delivered to the housing assembly 1, which will affect the phototherapy effect. Therefore, the phototherapy device can be used to treat patients with Alzheimer's disease who are restless and anxious.

The housing assembly 1 with open and loose design can make AD patients during various disease courses more willing to accept treatment. A single irradiation fraction can also last longer, such as 20 minutes, 30 minutes, or even longer each time, so as to further improve the treatment effect.

The embodiment of the present disclosure also provides a phototherapy apparatus for treatment of mental disease and mental disorder including depression, which includes the phototherapy device according to any embodiment of present disclosure. The phototherapy apparatus for treatment of mental disease and mental disorder including depression can use near-infrared light to treat mental diseases and mental disorders. It can supply cooled gas to the spaced cavity, which is formed between the housing assembly 1 and the user's scalp, through the first gas inlet 16 by the cooled gas supply mechanism 2, thus realizing the cooling of the user's head through the cooled gas supplied to the spaced cavity. It solves the problem that the heat generated by the irradiation assemblies 4 in the prior art causes the user's head discomfort. The present disclosure not only effectively improves the user's comfort and the phototherapy effect, but also improves the safety of the equipment, so as to avoid scalding the user.

In some optional embodiments, the average irradiance required for near-infrared light used by the phototherapy device can be determined and adjusted according to the user's attributes. For example, when the above phototherapy device is used to treat a disease that at least includes depression, the average irradiance is determined according to the light transmittance of the user's extracerebral tissue, so that the average irradiance for the user with low light transmittance of the extracerebral tissue is higher than the average irradiance for the user with high light transmittance of the extracerebral tissue.

In some embodiments, the phototherapy apparatus can also be used to treat autism, bipolar disorder and other cerebral functional related diseases requiring high average irradiance. Particularly, according to the type of disease or the age of the user, the cooled gas supply mechanism 2 can be used to make the head of each user in a more comfortable environment. For example, for elderly users with depression who have low sensitivity to temperature and pain and are afraid of cold, the cooled gas supply mechanism 2 can be used to make the temperature adjacent to the scalp of such users within the range from 25 degrees Celsius to 40 degrees Celsius when implementing phototherapy. For adolescent users with depression or adult users with depression who are not so sensitive to temperature and pain, the cooled gas supply mechanism 2 can be used to make the temperature adjacent to the scalp of such users within the range from 18 degrees Celsius to 37 degrees Celsius during phototherapy. For autistic children users who are more sensitive to temperature and pain, the cooled gas supply mechanism 2 can be used to make the temperature adjacent to the scalp of such users within the range from 18 degrees Celsius to 35 degrees Celsius during phototherapy.

Note that according to each control process in each embodiment of this application, for example, the control of the average irradiance of the near-infrared light emitted by the irradiation assembly 4, the control of the flow rate of the cooled gas supplied by the cooled gas supply mechanism 2, the control of the refrigeration function (temperature of the cooled gas) of the compressor, etc., can be implemented as computer executable instructions stored in the memory (or storage), and the corresponding steps can be implemented when the instructions are executed by the processor. It can also be implemented as hardware with corresponding logic computing capability. It can also be implemented as a combination of software and hardware (firmware). In some embodiments, the processor can be implemented as any of FPGA, ASIC, DSP chip, SOC (system on chip), MPU (such as but not limited to Cortex), etc. A processor may be communicatively coupled to a memory (or storage) and configured to execute computer executable instructions stored therein. The memory (or storage) may include read-only memory (ROM), flash memory, random access memory (RAM), dynamic random-access memory (DRAM) such as synchronous DRAM (SDRAM) or Rambus DRAM, static memory (such as flash memory, static random-access memory), etc., on which computer executable instructions are stored in any format. The computer executable instructions can be accessed by the processor, read from ROM or any other suitable storage location, and loaded into RAM for the processor to execute, so as to realize the wireless communication method according to various embodiments of the present application.

It should be noted that in each component of the system of this application, the components are logically divided according to the function to be realized. However, the application is not limited to this, and each component can be redivided or combined as needed. For example, some components can be combined into a single component, or some components can be further decomposed into more subcomponents.

Each component embodiment of this application can be implemented in hardware, or in software modules running on one or more processors, or in their combination. Those skilled in the art should understand that a microprocessor or a digital signal processor (DSP) can be used in practice to realize some or all functions of some or all components of the system according to the embodiments of the present application. The present application may also be implemented as an apparatus or device program (e.g., a computer program and a computer program product) for executing part or all of the methods described herein. Such a program implementing the present application may be stored on a computer-readable medium, or may be in the form of one or more signals. Such signals can be downloaded from Internet websites, or provided on carrier signals, or provided in any other form. In addition, the present application may be implemented by means of hardware including several different elements and by means of a properly programmed computer. In the unit claims listing several devices, several of these devices may be embodied by the same hardware item. The use of words first, second, and third does not indicate any order. These words can be interpreted as names.

Furthermore, although exemplary embodiments have been described herein, their scope includes any and all embodiments having equivalent elements, modifications, omissions, combinations (e.g., schemes crossed by various embodiments), adaptations or changes based on the present application. The elements in the claims will be interpreted broadly based on the language used in the claims, not limited to the examples described in the description or during the implementation of this application, and the examples will be interpreted as non-exclusive. Therefore, the specification and examples are intended to be considered as examples only, and the true scope and spirit are indicated by the following claims and the full scope of their equivalents.

The above description is intended to be illustrative rather than restrictive. For example, the above examples (or one or more of them) may be used in combination with each other. For example, those skilled in the art may use other embodiments when reading the above description. In addition, in the above specific embodiment, various features can be grouped together to simplify this application. This should not be interpreted as the intention that a public feature that does not require protection is necessary for any claim. On the contrary, the subject matter of the present application may be less than all the features of a particular disclosed embodiment. Thus, the following claims are hereby incorporated into the specific embodiments as examples or embodiments, wherein each claim is independently regarded as a separate embodiment, and it is considered that these embodiments can be combined with each other in various combinations or arrangements. The scope of the present application shall be determined by reference to the full scope of the appended claims and the equivalent forms empowered by these claims.

The above embodiments are only exemplary embodiments of this application and are not intended to limit this application. The protection scope of the application is defined by the claims. Those skilled in the art may make various modifications or equivalent substitutions to this application within the substance and protection scope of this application, and such modifications or equivalent substitutions shall also be deemed to fall within the protection scope of the application.

1. A phototherapy device for cerebral functional related diseases, comprising:
a headgear, including a housing assembly, inner side of which forms an accommodation cavity and is provided with a first gas inlet, the accommodation cavity accommodates the head of a user during phototherapy and forms a spaced cavity with scalp of the user, the spaced cavity is constructed to form gas pathway between the first gas inlet and the scalp of the user during phototherapy;
irradiation assembly, which is mounted to the housing assembly and transmits transcranial light to the inner side of the housing assembly; and
a cooled gas supply mechanism, which introduces cooled gas from outside of the housing assembly and is fluid-interconnected to the first gas inlet, so as to blow the cooled gas toward the scalp of the user via the first gas inlet and the spaced cavity sequentially during phototherapy.

2. (canceled)

3. The phototherapy device of claim 1, wherein, the headgear further comprises a support assembly provided to the inner side of the housing assembly, wherein the support assembly is constructed to maintain the spaced cavity between the housing assembly and the scalp of the user during phototherapy.

4. The phototherapy device of claim 1, wherein, the housing assembly comprises an outer housing, an euphotic inner housing, and a spacer, a chamber is formed between the outer housing and the inner housing, the spacer divides the chamber to form a first chamber and a second chamber, the irradiation assembly is provided within the first chamber, the first gas inlet is opened on the inner housing, the cooled gas supply mechanism is interconnected to the first gas inlet through the second chamber.

5. The phototherapy device of claim 4, wherein, it further comprising a hot gas pumping and discharging mechanism, which is interconnected to the first chamber and constructed to extract the hot gas heated by the heat generated by the irradiation assembly out and discharge it to the outside of the housing assembly.

6. The phototherapy device of claim 1, wherein, the headgear further comprises a branch pathway mechanism, which is provided to the inner side of the housing assembly and is fluid-interconnected to the first gas inlet, a first vent is opened onto the branch pathway mechanism, and the branch pathway mechanism includes a plurality of branch pathways in the spaced cavity.

7. The phototherapy device of claim 6, wherein, the headgear further comprises a support assembly provided to the inside of the housing assembly, the support assembly comprises one or more support bodies, each support body is opened with a second vent to be fluid-interconnected to the corresponding branch pathway, the cooled gas entering from the first gas inlet is discharged to the spaced cavity through the branch pathway and the second vent during phototherapy.

8. (canceled)

9. The phototherapy device of claim 4, wherein, the first chamber and the second chamber are both independent from each other.

10. The phototherapy device of claim 4, wherein, the top portion of the spacer is opened with a second gas inlet, which is constructed to introduce the cooled gas, the inner housing has an outer top surface, which forms a wind guide surface provided corresponding to the second gas inlet, so as to guide the cooled gas introduced via the second gas inlet to the periphery.

11. The phototherapy device of claim 10, wherein, the top portion of the inner housing is arch-shaped with a curvature less than a preset curvature, so that the introduced cooled gas diffuses gently to the periphery under the function of the arch-shaped top portion of the inner housing.

12. (canceled)

13. The phototherapy device of claim 5, wherein, a gas extraction port is opened in the outer housing, which is interconnected to the hot gas pumping and discharging mechanism and is provided with a retaining structure, wherein the retaining structure comprises a stop plate and a plurality of gas extraction holes interconnected to the first chamber, and the stop plate is provided corresponding to the gas extraction port in the up-down direction.

14. (canceled)

15. The phototherapy device of claim 1, wherein, it comprises a plurality of the irradiation assemblies, which are provided in array, and a plurality of the first gas inlets provided in groups, with each group of the first gas inlets corresponding to each individual irradiation assembly respectively.

16. (canceled)

17. The phototherapy device of claim 15, wherein, the area of inner housing for opening the each group of the first gas inlets comprises a first area covering the corresponding irradiation assembly and a second area surrounding the first area.

18. (canceled)

19. The phototherapy device of claim 1, wherein, during phototherapy the gas pathway formed by the spaced cavity enables the wind velocity adjacent to the scalp of the user to be maintained within the range from 0.5 m/s to 3.5 m/s.

20. The phototherapy device of claim 1, wherein, the temperature of the cooled gas is tuned, so that during phototherapy the temperature adjacent to the scalp of the user maintains within the range from 23 degrees Celsius to 40 degrees Celsius in case that the wind velocity adjacent to the scalp of the user is maintained within the range from 0.5 m/s to 3.5 m/s.

21. The phototherapy device of claim 1, wherein, when the phototherapy device is used for treating Alzheimer's disease, the housing assembly accommodates loosely the user's head, allowing the head to rotate within a predetermined angle range and move in the up-down direction within a predetermined distance range during the treatment.

22. The phototherapy device of claim 1, wherein, the irradiation assemblies are constructed to emit near-infrared light with an average irradiance greater than 40 mW/cm² towards the patient's head, and the temperature near the patient's scalp is within the range from 18 degrees Celsius to 43 degrees Celsius during the phototherapy.

23. The phototherapy device of claim 1, wherein, the housing assembly forms a cooled gas transmission inner cavity, which is fluid-interconnected to the first air inlet.

24. The phototherapy device of claim 4, wherein, the first chamber is located outer than the second chamber.

25. The phototherapy device of claim 3, wherein, the cooled gas supply mechanism introduces the cooled gas from an upperside of the housing assembly to the cooled gas transmission inner cavity, which forms a wind guide surface to guide the cooled gas to the periphery and then downwards.

26. The phototherapy device of claim 1, wherein, the phototherapy device is used for treating at least one of Alzheimer's disease, depression, autism, and bipolar disorder.
